(19) United States
(12) Patent Application Publication
(10) Pub. No.: US 2018/0154072 A1
(43) Pub. Date: Jun. 7, 2018

(54) MEDICAL DEVICE AND METHOD FOR TEMPERING INFUSION FLUIDS IN A RACK-LIKE STRUCTURE

(71) Applicant: SEIRATHERM GMBH, Herzogenaurach (DE)

(72) Inventors: Matthias ROTH, Giggenhausen (DE); Thomas REICHTHALHAMMER, Unterschleissheim (DE)

(73) Assignee: SEIRATHERM GMBH, Herzogenaurach (DE)

(21) Appl. No.: 15/737,655

(22) PCT Filed: Jun. 20, 2016
(86) PCT No.: PCT/EP2016/064170

§ 371 Date: Dec. 18, 2017

(30) Foreign Application Priority Data
Jun. 26, 2015 (EP) 15001932.1

(51) Int. Cl. A61M 5/14; A61M 5/00

(52) U.S. Cl. CPC A61M 5/1415; A61M 5/008; A61F 2007/126; A61M 2205/3334; A61M 2205/3372; A61M 5/1413

(57) ABSTRACT

The present invention relates to a method and medical device for tempering an infusion fluid for hypothermia purposes comprising a frame for introducing the device into one or more slots of a rack of given dimension and/or shape, the frame comprising at least one, preferably at least two rail(s) and/or hook(s) for inserting the device into the rack and at least one releasable lock for releasably locking it therein. Furthermore, at least one connector is provided for connecting the device with a central unit for controlling and/or monitoring the device which is adapted to also control and/or monitor at least a further medical device. At least one incoming port is connected to at least one source of infusion fluid adapted to contain infusion fluid during use. At least one pump is also provided for pumping the infusion fluid from the incoming port to an outgoing port, and at least one tempering device adapted to cool and/or heat the incoming infusion fluid to a preset temperature and/or temperature profile for delivering it to the outgoing port.

### FIELD

The invention is directed to a medical device and method for tempering an infusion fluid, particularly for hypothermia treatment, in a rack-like structure.

### BACKGROUND

Hypothermia is usually called a condition in which the body's core temperature drops below that required for normal metabolism and body functions. This is generally considered to be less than 35.0° C. (95.0° F.). Characteristic symptoms depend on the temperature. Targeted temperature management (TTM) previously known as therapeutic hypothermia or protective hypothermia is active treatment that tries to achieve and maintain a specific body temperature in a person for a specific duration of time in an effort to improve health outcomes. This is done in an attempt to reduce the risk of tissue injury from lack of blood flow. Periods of poor blood flow may be due to cardiac arrest or the blockage of an artery by a clot such as may occur in stroke. Targeted temperature management improves survival and brain function following resuscitation from cardiac arrest. Evidence supports its use following certain types of cardiac arrest in which an individual does not regain consciousness. Targeted temperature management following traumatic brain injury has shown mixed results with some studies showing benefits in survival and brain function while other show no clear benefit. While associated with some complications, these are generally mild. Targeted temperature management can advantageously prevent brain injury by several methods including decreasing the brain's oxygen demand, reducing the proportion of neurotransmitters like glutamate, as well as reducing free radicals that might damage the brain. The lowering of body temperature may be accomplished by many means including the use of cooling blankets, cooling helmets, cooling catheters, ice packs and ice water lavage.

Medical events that targeted temperature management may effectively treat fall into five primary categories: neonatal encephalopathy, cardiac arrest, ischemic stroke, traumatic brain or spinal cord injury without fever, and neurogenic fever following brain trauma.

According to EP 2010 739 239 a hypothermia system comprises a fluid reservoir, a heat exchanger assembly, a catheter in fluid communication with the fluid reservoir, and a pump system configured to infuse hypothermic fluid into a patient cavity and extract hypothermic fluid from the patient cavity. The hypothermia system can infuse and extract fluid automatically from the patient cavity. In one embodiment, the patient cavity is a peritoneal cavity. A safe access device to gain access to the patient cavity is also provided. This, however, provides a rather voluminous system and makes it necessary to access a patient's cavity with a number of risks.

The applicant's WO 2009/056640 A2 describes an adjustment and stabilization of the body temperature of a patient. This is achieved by an actively controlled infusion of fluid of a preferably known and/or controlled temperature, employing a feedback control with body temperature being a measured variable and fluid flow being an actuating variable. According to a particularly preferred embodiment, the adjustment and stabilization of the body temperature of a patient is achieved by an actively controlled balance of volume flows of infusion fluids provided at temperatures differing from one another. The volume flows result in a combined volume flow at a suitable temperature being continuously infused into the patient. This document is herewith incorporated by reference.

According to a scientific paper in the periodical “Medizintechnik”, 1/96, pages 7 to 11 a need exists to standardize the different components used in hospitals and being placed next to patients for the delivery of different medications. Moreover, racks with a plurality of components for medical devices become more and more used. Typically they offer a slot for introducing medical devices such as syringes or pumps pumping infusion fluids with different medicaments for their intravenous delivery. Further in U.S. Pat. No. 4,756,706 a central processing unit is described for centrally controlling and monitoring the different components mentioned before. In U.S. Pat. No. 5,376,070 a communication controller is suggested for interchangeably connecting and disconnecting different components. U.S. Pat. No. 5,681,285 further suggests a central storage for selecting medication relevant data from a medication register. U.S. Pat. Nos. 4,978,335, 5,317,506 as well as U.S. Pat. No. 5,609,575 are directed to the further automatization of reading information from medications carriers or for calculating delivery rates. All these documents are herewith incorporated by reference.

WO 02/26286 A2 describes an extracorporeal blood perfusion system includes a disposable assembly and a control unit having a control interface region. The interface region includes pump assemblies for selective pumping of venous blood, arterial blood, cardioplegia solution, suctioned blood and blood removed from the left ventricle. Valve assemblies control the flow of fluids through the assembly and to/from the patient and sensors monitor various fluid parameters including temperature and pressure within the various fluid circuits. The user interface is a functional screen interface for effecting the operation of the control unit and valve assemblies. The screen interface may be a touch screen having objects that corresponds to the component interface region. The display may be selectively controlled to provide graphic depictions of disposable assembly components with corresponding narrative instructions.

WO 2013/102495 concerns an arrangement of a rack and a medical device to be attached to the rack, the rack comprises a first connection element and the medical device comprises a second connection element which in an attached state of the rack and the medical device is releasably connected to the first connection element of the rack to establish an electrical connection between the medical device and the rack. The first connection element and the second connection element each comprise at least two electrical contacts, wherein in the attached state of the rack and the medical device via the at least two electrical contacts both a low speed data connection and a high speed data connection between the medical device and the rack is established. Thus, an arrangement of a rack and a medical device is provided which allows for an easy attachment of the medical device to the rack by providing a secure and reliable and at the same time versatile electrical connection between the medical device and the rack. This document is incorporated herewith by reference.

EP 0 960 627 A2 is directed to an assembly for a central control and/or monitoring of infusion pumps has defined positions to mount the infusion pumps, each with an interface for the data communications at the infusion pump connections. The central control has a display to show the total condition of the infusion pumps, with the display layout matching the pump positions. An interface for the energy supplies is also at the pump mounting positions, linked to a common energy supply at the central control. The infusion pumps and the central control also have an emergency energy supply, to ensure continuing operation in the event of a power failure. The central control and the infusion pump assembly are in separate units that are joined together, or are separated. The control sub-unit gives an automatic control function for the infusion pumps and/or the communication link between the pumps and the superior unit. On the insertion of the infusion pumps into the mounting and carrier system, the mechanical lock also gives the interface link for data communication with the central control. When an infusion pump is removed, the disconnection is detected and is further processed independently in a given function or the previous condition is maintained, or it is transferred into a safe condition. The mounting system for the infusion pumps can also be fitted with further modules to register physiological patient parameters, linked to the central control. The central control is also linked to other units within the patient's environment for data exchange, or it is connected to another relatively non-mobile medical system. The control has a data input, for the insertion of parameters to be used for the control of the infusion pumps. The parameters can include the concentration of a physiologically active substance within the infused fluid and/or the patient's body weight and/or a required concentration of the active substance in the patient's blood. One or more monitors measure physiological parameters, which can be used to affect the infusions. The central control and/or monitor has an index of medications, inserted manually or taken from another assembly in relation to patient parameters, to be stored. They can be altered manually the condition of each infusion pump is displayed at the central unit, and especially alarms. Also this document is herewith incorporated by reference.

### SUMMARY

The problem underlying the present invention is to provide an improved or ameliorated cooling device, cooling system and/or cooling method for infusion fluids.

The problem can be solved by the subject matter of the present invention exemplified by the description and the claims.

The present invention is directed to a medical device for tempering an infusion fluid for hypothermia. A frame is provided and assembled and/or adapted to introduce the device into one or more slots of a rack of given dimension and/or shape. The rack can be of any shape, either with a single post for assembling the medical device and other medical devices and/or a shelf-like structure with more than one post and/or at least one or more walls. Moreover, the rack can be adapted to allow several medical devices to be placed in at least one vertical and/or at least one horizontal row(s). The frame of the medical device comprises at least one, preferably at least two rail(s) and/or hook(s) for inserting the device into the rack. In case the rack comprises essentially one post one or more hooks can be provided. In case of a shelf-like rack one or more rail(s) can be provided for slidingly placing or allowing the device to be placed in the rack. The rail(s) and/or the respective counterpart(s) at the rack can be of any known structure with sliding or bearing-supported structure(s) and/or of expandable and/or telescoping nature. Moreover, the medical device can comprise at least one releasable lock for releasably locking the device in the rack. This can prevent the accidentally removing of the medical device in or at the rack and/or the defined position of the device and/or its other components interfering with the rack and other elements. The term locking does not necessarily mean that a specific element is provided it can also be enabled by a mechanical and/or electronic indicator indicating the defined position of the medical device at and/or in the rack.

Moreover, the medical device can comprise at least one connector for connecting the device with a central unit for controlling and/or monitoring the device. The central unit can be adapted to also control and/or monitor at least a further medical device. The central unit can be located in, at and/or distant from the rack. Sometimes central units remotely located from the rack or even the room the rack is located in allow a centralized control and/or monitoring of the rack and/or the medical devices contained therein. Such centralized control and/or monitoring can be arranged in a specific area or room being used for the control and/or monitoring such as by specifically trained staff. The medical device can nevertheless have also its own independent or dependent control and/or monitoring elements, such as at least one CPU, circuitry, storage, monitor, keyboard etc.

The medical device can also comprise at least one incoming port that is or can be connected to at least one source of infusion fluid adapted to contain infusion fluid during use. The term port comprises any direct connection and/or any socket, connector etc. for a pipe, duct etc. to the infusion fluid and/or expelling container and/or patient. A plurality of sources of infusion fluid and/or a plurality of incoming ports can also be provided. On top of the medical device one, two or more sources for infusion fluid can be assembled, depending on the needs.

The medical device can also comprise at least one pump for pumping the infusion fluid from the incoming port to an outgoing port. One pump for each port or for more than one port can be also provided.

The medical device can also comprise at least one tempering device adapted to cool and/or heat the incoming infusion fluid to a preset temperature and/or temperature profile for delivering it to the outgoing port. The tempering device can be of any known kind and can use or utilize conportion, radiation, electromagnetic, resistance, or cycle refrigeration and/or any other known tempering elements.

The tempering device and/or the pump can be located outside, in and/or within the frame. The tempering device can be a unitary device or split with at least one element in or within the frame and at least one element out of the frame. In case one part is out or the frame this part can be the heavier part such as the cooling part of the tempering device. If it or a part thereof is located outside the frame the infusion fluid can be lead to it from the part of the medical device being located in or at the rack.

The medical device's frame in the rack can define at least one front side in use and at least one rear side opposite or away from the front side further with at least one cooling assembly for cooling the tempering device, the cooling assembly being located distant from the front side of the rack and/or at or adjacent the rear side.

The cooling assembly can comprise at least one heat exchanger and/or at least one ventilator whatever is needed to sufficiently heat and/or cool the infusion fluid(s). The heat exchanger and/or ventilator can be located at and/or adjacent the rear side of the rack and can be adapted to convey any air away from the front side towards the back side and further out of the device through and/or away from the back side.

The medical device can comprise at least one first sensor for measuring the/each flow rate and/or temperature of the infusion fluid(s) during use. The temperature measurement can comprise the measurement of the temperature before or after the tempering and/or of one or more fluid(s) that are not tempered in the medical device but exclusively or in part delivered from other devices in the rack or associated therewith. These measurements can be used in order to determine or align and/or better control the tempering of at least one fluid and/or the hypothermia treatment of a patient.

Further, at least one flow rate controlling assembly for controlling the flow rate(s) of the infusion fluid(s) during use can be controlled and/or adjusted. The controlling assemblies can be any device for opening and/or closing any part(s) of the ports, such as by valves.

The flow rate sensor(s) and/or the temperature sensor(s) and the flow rate controlling assembly(/ies) and/or tempering device(s) form at least part of a closed-loop controlling unit. Thus, any measurement can result in a respective flow rate and/or tempering adaption. The sensor(s) mentioned above and below can be used for measuring an fluid upstream, in or downstream of the medical device individually or combined in any manner.

A flow block arranged at and/or in the medical device can have at least one passage for the infusion fluid and at least a further infusion fluid. Each fluid can be treated by the medical device individually or jointly. E.g., the passage can be either adapted to allow two incoming ports to flow through the medical device individually, together and/or connected to one passage or a group of passages. The passage(s) can at least two infusion fluids within the flow block, upstream and/or downstream thereof.

The flow block can be arranged at or adjacent the front side of the device and/or can be composed from individual modules for different infusion fluids and/or combinations thereof, the modules being releasably or non-releasably connectable to each other.

At least one sensor for measuring the flow rate and/or temperature of any infusion fluid or combined fluids is attached to each module and can be modularly connected or reported to the central unit.

At least one sensor for measuring gravity and/or acceleration for a proper position of the device, particularly in a rack, and/or at least one sensor for sensing leakage at least of one of gas and/or fluid can also be provided.

The present invention is also directed to any method according to any one of the principles and/or embodiments described before or below.

The flow rate and/or temperature of at least two infusion fluids can be individually and/or jointly measured and/or individually and/or jointly controlled.

All aspects of the present invention are adjusted to operate or be operated without a patient. According to one aspect of the present invention the infusion fluid can be collected by a container or can be infused into a patient.

The present invention can provide a standardized assembly or device for hypothermia to be used and implemented in an advantageous fashion with other related and/or non-related assemblies or devices. Central controlling and monitoring can also be used in order to have one or a common usability with other assemblies and/or devices.

### DRAWINGS

The skilled person will understand the drawings, described below, are for illustration purposes only. The drawings are not intended to limit the scope of the present teaching in any way.

FIG. 1 exemplifies a rack with a medical device according to one aspect of the invention;

FIG. 2 exemplifies an upper view onto some elements within the medical device according to one aspect of the invention;

FIG. 3 exemplifies a front view onto some elements within the medical device according to one aspect of the invention; and

FIG. 4 exemplifies a controlling scheme for the medical device.

FIG. 1 shows an example of a rack 1 for arranging a medical device 20 or a plurality of medical devices during use. Other racks can also be used, such as with open or slightly more open sides, just one or more major column(s) for holding devices, for holding devices with other dimensions or other arrangements of medical devices, such as some of them being located side by side and/or below and/or above each other. In the present example at least posts 2 or walls 2 at the two sides are shown. At and/or in the posts 2 or walls 2 are rails 3 for holding any medical devices such as the device 20 shown. In dotted lines another medical device is shown in the upper part of the rack 1. The rails 3 can be adapted and/or arranged in order to allow the medical devices to be sled into the rack 1. Self-expanding, self-telescoping and/or sliding rails can be used. The medical devices can be sled in from the front. A lock for securing the medical device (20) can be provided (which is not shown).

At least one source 10 of an infusion fluid can be mounted directly at or above the rack 1. A plurality of sources 10 of the same or different infusion fluids can also be attached to the rack. The source(s) 10 can be alternatively and/or additionally attached to a different device apart from the rack with ports from the source(s) being brought into fluid connection with at least one of the medical devices within or at the rack 1.

According to FIG. 1 a port 11 (which is for later purposes named incoming port 11) delivers the infusion fluid to a medical device 20 that in the example shown in FIG. 1 is mounted over two rack positions at the bottom thereof. Any other shape, extension, dimensions etc. of the medical device can also be used. The medical device 20 can have a display 21. The display may be omitted when there is another central controlling unit either in, at or adjacent the rack or in a remote position such as in a monitoring room. Further, a keypad 22 can be also arranged. Instead, the display may be a display also acting as a touch panel with a capacity activated or pressure activated cover panel. The activation can also take alternatively or additionally place from a remote position.

FIG. 1 moreover shows that another port 12 which is called outflowing port 12 which can be provided to further deliver the infusion fluid(s) to a container, such as for test purposes, or to a patient.

A flow block 30 is also shown. The incoming port 11 can be directed into the medical device by a respective inflow section 30a of the flow block 30. In the example shown the infusion fluid is then directed further into the interior of the medical device and can be directed out of the medical device at an outflow section 30a′. At the flow block at the inflow section 30a and/or the outflow section 30a′ a temperature and/or flow rate sensor can be arranged. Instead of or additionally to the flow rate sensor also at least one dosage pump can be placed, such as a peristaltic pump.

Further section(s) 30 b, b′, c, c′, d, d′ can be also arranged. The number and sort of these sections can vary upon needs and can be assembled in a modular form. They can be clicked or secured to each other and/or a number of sections can already be arranged. They can also vary, as will be further apparent from below and the figures. In the example shown in FIG. 1 an infusion fluid can be introduced into interior elements of the medical device at section 30 a, a′. At section 30d, d′ an already existing connection between the inflow and the outflow section is shown which will allow a fluid just to pass these sections without being detoured into other elements of the medical device 20. This may be useful when an infusion fluid is sensitive to any tempering or rather small in volume. Anyhow, one or more temperature sensor(s) (not shown) in or at sections 30d, d′ can measure the temperature and/or at least one flow rate sensor(s) can measure the flow rate of the respective fluid. This may help in determining the amount and/or temperature of other fluids in order to optimize the temperature and/or flow rates of other fluids for hypothermia or hyperthermia purposes.

FIG. 2 allows a look into the medical device 20 from an elevated position. The medical device has a frame 28 defining at least part of the outer contour of the medical device 20 and can comprise a casing 28 or body. Also rails 26 can be provided in order to allow the medical device to be sled into the rack (not shown). A lock is also not shown in any of the figures. A plug or connector 27 is shown for connecting the medical device 20 to current and/or a central controlling unit (not shown). Further connectors can be arranged for slow and fast speed connections, different standards etc. The connector may also be a wireless connection device which may be activated by the medical device being placed in the rack.

A controller 23 can be arranged in the medical device for the local controlling of the device and for other controlling purposes. A heat exchanger 24, such as a heat exchanger comprising a ventilator 24, can be arranged in the back part of the medical device 20. Also an alternative or additional arrangement of a heat exchanger can be placed at or in the vicinity of the side and can be preferably be directed away from any patient and/or user. The heat exchanger 24 is connected to a tempering device 25.

From FIG. 2 it is apparent that fluids from sections 30a and 30b can be combined before they enter the tempering device 25. This may be done when both fluids can be or shall be mixed or when a spare source of infusion fluid is connected to section 30b. They are further transported by a pump 33a to the outflow section. There they can leave the medical device in section 30a′ and/or 30b′, depending on the needs. As another or an additional example the fluid in section 30c enters the tempering device 25 without being combined with another fluid. It is conveyed by the pump mentioned before or another pump 33c and pumped to the respective outflow section.

The tempering device 25 can be any tempering device for cooling and/or heating infusion fluids. The tempering device 25 can comprise or utilize conduction, radiation, electromagnetic, resistance, or cycle refrigeration based and/or any other tempering elements.

FIG. 3 shows a view from the front into the medical device 20. The same elements have the same reference numerals as used before. The arrangement and presence of elements can vary even considerably from the embodiment shown. In the tempering device 25 the inflow ports or apertures 31, 31′ and the outflow ports or apertures 32, 32′ are shown. At least one of them can be provided or a larger number of them, whatever is needed.

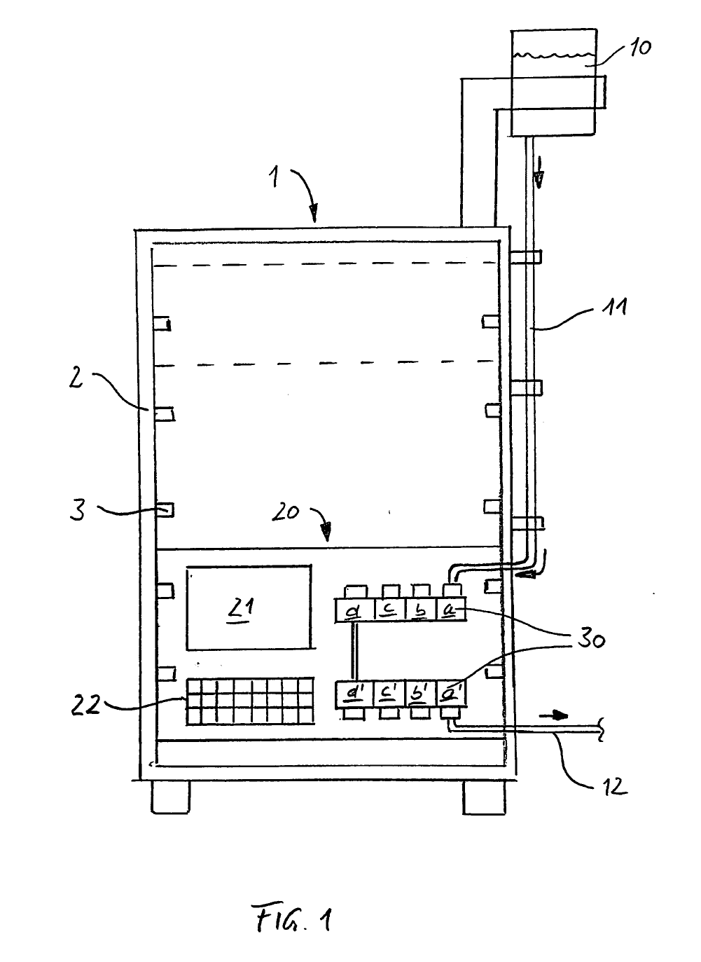

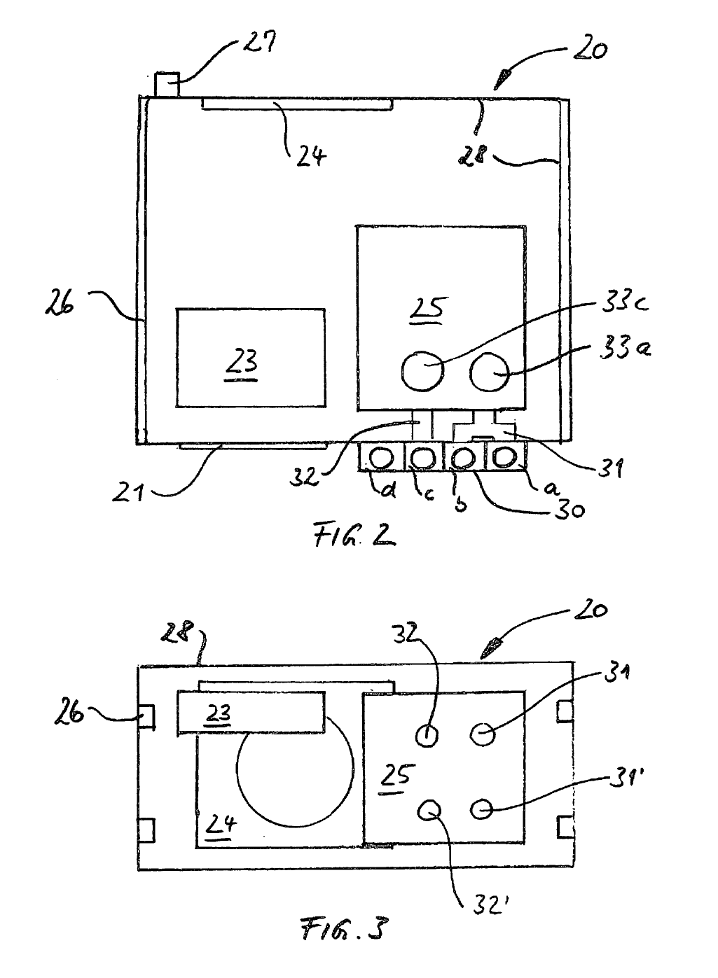

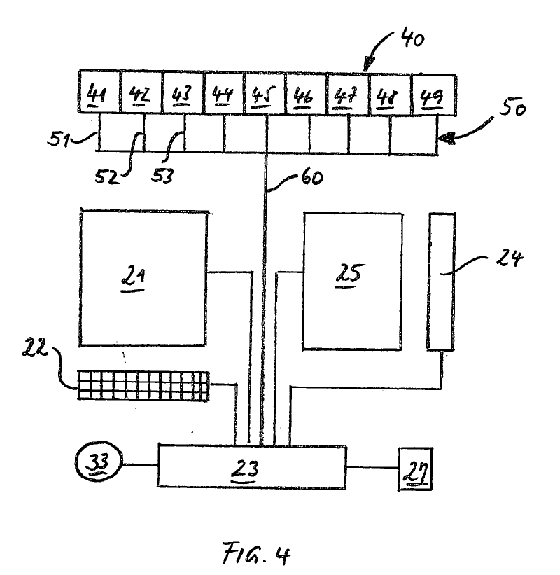

FIG. 4 shows one example for a controlling scheme for the medical device. A controller 23 can be connected and/or communicate one- or bi-directionally with the elements to be controlled. The connections can be realized by CAN-bus structure, Flex Ray, MOST, AS-I and/or wireless. The controller communicates with at least one or a set 40 of sensors 41 to 49. In FIG. 4 the number of sensors is of exemplifying nature only. Besides flow rate and/or temperature sensors mentioned above and below also other sensors can be used, such as gravity and/or acceleration sensors for assuring a proper position of the medical device within the rack, gas and/or liquid sensors for ensuring the early detection of leaking at least of one of gas and/or liquid. As mentioned before, the sensors can be located upstream and/or downstream the tempering device 25 or at any appropriate position within, at or out of the medical device.

The set 40 of sensors or individual sensors 41 to 49 can be connected by a set 50 of communication and/or controlling lines with the controller 23, and/or grouped or together by a common communication line 60.

The controller 23 can control the monitor 21 and/or keyboard 22. In case the monitor is a touch sensitive monitor the controller 23 can also control this. The controller 23 can also control the tempering device 25 and/or a cooling assembly 24. At least one of the sensors 40 can also be a temperature sensor for sensing the temperature within the medical device and may activate the cooling assembly 24 also independently of the tempering device in case the temperature is reaching or over a threshold value. The cooling assembly 24 can also cooperate with the tempering device by cooling a heat exchanger cooperating with the tempering device 25.

The controller can also control the at least one pump 33 and/or the connector 27. The connector 27 with the controller 23 can communicate with any external controlling and/or monitoring device. As mentioned before, the connector can also be a wireless connector. At least one extra interface (not shown) can also be provided and/or attached to the controller 23 for accessing the controller with other means, such as diagnoses tools etc.

Thus, it has been found that the present invention and aspects thereof can deliver a more centralized, standardized, safer and/or faster and further preferably more precisely adjusted or positively controlled temperatures of the infusion fluid. Thus, more individualized and a better adjusted flow of infusion fluids can be realized or a patient can be treated more according to the needs detected in real time or close to real time.

While the disclosure has been illustrated and described in detail in the drawings and foregoing description, such illustration and description are to be considered illustrative or exemplary and non-restrictive; the disclosure is thus not limited to the disclosed embodiments. Variations to the disclosed embodiments can be understood and effected by those skilled in the art and practicing the claimed disclosure, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to fulfill aspects of the present invention. The present technology is also understood to encompass the exact terms, features, numerical values or ranges etc., if in here such terms, features, numerical values or ranges etc. are referred to in connection with terms such as “about, ca., substantially, generally, at least” etc. In other words, “about 3” shall also comprise “3” or “substantially perpendicular” shall also comprise “perpendicular”. Any reference signs in the claims should not be considered as limiting the scope.

1. Medical device for tempering an infusion fluid for hypothermia purposes comprising:
a. a frame for introducing the device into one or more slots of a rack of given dimension and/or shape, the frame comprising at least one, preferably at least two rail(s) and/or hook for inserting the device into the rack and at least one releasable lock for releasably locking it therein;
b. at least one connector for connecting the device with a central unit for controlling and/or monitoring the device which is adapted to also control and/or monitor at least a further medical device;
c. at least one incoming port being connected to at least one source of infusion fluid adapted to contain infusion fluid during use;
d. at least one pump for pumping the infusion fluid from the incoming port to an outgoing port, and
e. at least one tempering device adapted to cool and/or heat the incoming infusion fluid to a preset temperature and/or temperature profile for delivering it to the outgoing port.

2. Medical device according to claim 1, wherein the tempering device and/or the pump is/are located within the frame.

3. Medical device according to claim 1, wherein the frame in the rack defines at least one front side in use and at least one rear side distant, opposite and/or away from the front side, the device further comprising at least one cooling assembly for cooling the tempering device, the cooling assembly being located distant from the front side of the rack and/or at or adjacent the rear side.

4. Medical device according to claim 1, wherein the cooling assembly comprises at least one heat exchanger and/or at least one ventilator, the heat exchanger and/or ventilator being located at and/or adjacent the rear side of the rack and being adapted to convey air away from the front side towards the back side and/or in a direction out of the frame through and/or away from the backside.

5. Medical device according to claim 1, the device further comprising at least one sensor for measuring the flow rate and/or temperature of the infusion fluid(s) during use.

6. Medical device according to claim 1, the device further comprising at least one flow rate controlling assembly for controlling the flow rate of the infusion fluid during use.

7. Medical device according to claim 1, wherein the flow rate sensor(s) and/or temperature sensor(s) and the flow rate controlling assembly and/or the tempering device form at least part of a closed-loop controlling unit.

8. Medical device according to claim 1, the device further comprising at least one sensor for measuring the temperature and/or flow rate of at least a further second infusion fluid.

9. Medical device according to claim 1, wherein the medical device comprises a flow block having at least one passage for the infusion fluid and at least one further infusion fluid, the passage either being adapted to allow two incoming ports to be connected to the one passage or the passage being a merged passage merging the at least two infusion fluids within the flow block.

10. Medical device according to claim 9, wherein a third sensor is provided and adapted to measure the flow rate and/or temperature of the combined infusion fluids.

11. Medical device according to claim 9, wherein the flow block is arranged at or adjacent the front side of the device.

12. Medical device according to claim 9, wherein the flow block comprises individual modules for different infusion fluids and/or combinations thereof, the modules being releasably connectable to each other.

13. Medical device according to claim 1, wherein at least one sensor for measuring the flow rate and/or at least one sensor for controlling the temperature of any infusion fluid is attached to each module and modularly connectable to the central unit.

14. Medical device according to claim 13, wherein at least one sensor for measuring gravity and/or acceleration for a proper position of the device, particularly in a rack, and/or at least one sensor for sensing leakage at least of one of gas and/or fluid.

15. Method of tempering an infusion fluid for hypothermia purposes, particularly with a medical device according to claim 1, comprising the following steps:
a. providing a frame with at least one, preferably at least two rail(s) and/or hook(s) for inserting the device into a rack and at least one releasable lock(s) for releasably locking it therein;
b. introducing the device into one or more slots of a rack of given dimension and shape;
c. connecting the device by introducing it into the slot with a central unit for controlling and/or monitoring the device, the central unit also controlling and/or monitoring at least a further medical device;
d. coupling an incoming port being connected to at least one source of infusion fluid adapted to contain infusion fluid during use;
e. pumping the infusion fluid from the incoming port to an outgoing port by at least one pump; and
f. providing cooling and/or heating the incoming infusion fluid to a preset temperature and/or temperature profile by at least one tempering device and delivering it to the outgoing port.

16. Method according to claim 15, wherein the flow rate and/or temperature of at least two infusion fluids are individually or jointly measured.

17. (canceled)

18. Method according to claim 15, wherein the flow rate and/or temperature of at least two infusion fluids are individually or jointly at least one of measured and controlled.
